(19) United States
(12) Patent Application Publication
(10) Pub. No.: US 2007/0225780 A1
(43) Pub. Date: Sep. 27, 2007

(54) MAGNETIC THERAPY DEVICE

(76) Inventor: Emilio Paez, Fresh Meadows, NY (US)

Correspondence Address: ADVANTIA LAW GROUP, 9035 SOUTH 1300 EAST, SUITE 200, SANDY, UT, 84094, US

(21) Appl. No.: 11/277,449

(22) Filed: Mar. 24, 2006

(51) Int. Cl. A61N 1/00

(52) U.S. Cl. 607/103

(57) ABSTRACT

A magnetic device for therapy of humans and/or animals that may include one or more magnetic flux emitters. The emitter(s) may include one or more apertures. The emitter(s) may include one or more protective layers in the aperture(s). The emitter(s) may include one or more indicators to indicate orientation(s) of flux. The emitter(s) may be toroidal in shape and/or in any other shape. The emitter(s) may be permanent magnet(s). The magnetic device may include a magnetic therapy system for beneficial use of a person or person(s) and/or other users. The device may include one or more reclining surfaces. The device may include one or more supporting legs coupled to and supporting the reclining surface(s). The device may include one or more magnetic flux emitters. Each emitter may be located about one or more of the supporting legs in any combination. Each of the emitters may be oriented in substantially the same direction and/or oriented in a variety of directions. The reclining surface may be a bed. The emitter(s) may include one or more protective layers to prevent damage to the supporting leg(s).

### BACKGROUND OF THE INVENTION

1. Field of the Invention

The present invention relates generally to magnetic therapeutic methods, and, more particularly, there are annular magnetic device(s) placed about bed legs to generate a magnetic field around the bed to promote health benefits to user(s).

2. Description of the Related Art

There are a variety of treatments and health-care methods and devices that involve the application of magnetic fields to animals and humans. For example, such devices include magnetic watches, rings, beds, vests, belts, shoes, etc. These devices and methods are all available on the market and share the feature of applying magnetism locally on the body for treatment and health-care.

The following patents are incorporated by reference for their supportive teachings and for illustrating features of the prior art:

U.S. Pat. No. 5,788,624, entitled Magnetic Therapy and a Magnetic-Field Scanning Physiotherapeutic Device, discloses such a magnetic device. Specifically, there is a magnetic system and physiotherapeutic device performing such a magnetic therapy. The therapy involves the application of magnetism that provides a transient penetration of magnetic flux by means of a magnetic field scanning of a whole body or a portion thereof in a three-dimensional gradient magnetic field. The physiotherapeutic treatment device comprises: (1) a bed; (2) two magnets straddling each side of the bed; (3) a mechanical-electrical transmission system; and (4) an electric control system. The therapeutic and health-care effect is achieved by means of magnetic-field scanning of the human body while the body is lying on the bed facing upward, while the 2 magnets, at properly adjusted heights and actuated by the control panel, move horizontally along the body. Clinical tests demonstrate this invention has remarkable therapeutic effects upon the treatment of a variety of disorders.

U.S. Pat. No. 5,084,003, entitled Magnetic Massage Therapy Device, also discloses such a magnetic device. Specifically, it is a massage device having a low-frequency voltage pulse generator (IG), which supplies an electromagnetic field coil (MZ), inside of which a human or animal body is placed, on the surface of which a magnet cover or magnet apron (MS) is located inside the field coil (MZ) and which is comprised of small permanent magnet magnetic plates (PM) which are flexibly connected with each other, so that these small magnetic plates (PM) exert a massaging effect on the surface of the body, because they are excited by means of the low-frequency electromagnetic field into pivoting oscillations. The massage effect stimulates the flow of blood and draining of lymphatic fluid and increases the effect of the electromagnetic low-frequency treatment.

U.S. Pat. No. 6,203,486 B1, entitled Earth Magnetic-Field Augmenters, discloses using magnetic-field-augmented beds, mattresses, seating furniture, desks, carrels, railings, rooms, and buildings include magnetic-field augmenters that, when connected to a source of electrical power, provide a healthful magnetic field for entire bodies of human beings or other living things in living spaces. In another embodiment, a magnetic-field augmenter is hung on a wall, and in another embodiment, a magnetic-field augmenter is arranged on a floor around seating furniture. Included among the various types of magnetic-field augmenters are bundled augmenter coils, two-axis augmenters, open-sided augmenters, and two-axis open-sided augmenters. A protective device protects against loose connections and shorts.

U.S. Pat. No. 5,667,469, entitled Strong Magnetism Therapeutic Apparatus with Permanent Magnets Rotating at Low Frequency, discloses using a strong magnetism therapeutic apparatus with permanent-magnet sets rotating at low frequency in which a base set made of magnetic material is fixed on a plate capable of rotating in both direction and at least two adjacent permanent-magnet sets for producing strong magnetic field are secured to said base set and separated by a isolating block made of non-magnetic material, A pole head is secured to the upper surface of each permanent-magnet set. The penetrating depth within the object to be treated is up to 500 mm. When used as a therapeutic instrument, this apparatus can produce desirable analgesic effect for late patient of, for example, cancer without the minus effect of radiation therapy and chemotherapy. The apparatus has also wide application in biologic and chemical field.

U.S. Pat. No. 4,509,219, entitled Sleeping Mattress, discloses using a sleeping mattress that comprises in order a first layer made of cushion material formed with wave-like projections, a second layer having semi-circular projections provided with permanent magnets, a third layer stuffed with palm fibers and reinforcing materials, and a fourth layer composed of cushion materials. A magnetic curing effect by the magnets and a spine balancing effect by moderate hardness are obtained during sleeping, and comfortable sleeping may be achieved.

U.S. Pat. No. D445,195, entitled Medical Magnet, discloses using an ornamental design for a medical magnet.

It is theorized that magnetic therapeutic devices may provide the following benefits to humans and/or animals: Overall well-being mentally, emotionally, spiritually, and/or physically; feeling better about oneself, increased likelihood to exercise, eat better, and loose weight, manage one's weight, body fat, and/or hunger; the neutralizing of body energy, the repelling of negative thoughts, the saving and strengthening of personal relationships; the enabling of sick humans or animals to heal and be cured; and/or the benefit of greater productivity and creative output for the user(s). However, the magnetic therapeutic device may play other roles, and therefore this theory is not intended to be limiting to the invention.

What is needed is a magnetic therapy device that solves one or more of the problems described herein and/or one or more problems that may come to the attention of one skilled in the art upon becoming familiar with this specification.

### SUMMARY OF THE INVENTION

The present invention has been developed in response to the present state of the art, and in particular, in response to the problems and needs in the art that have not yet been fully solved by currently available magnetic therapy devices. Accordingly, the present invention has been developed to provide a system for using magnetic device(s) placed about bed legs to generate a magnetic field around the bed to promote health benefits to human and/or animal users.

In one embodiment of the invention, the magnetic therapy device may include a magnetic flux emitter with an aperture therethrough; and/or a protective layer disposed in the aperture. The protective layer may be a soft material to protect the bed against damage. The emitter may also have an indicator configured to indicate the orientation of flux. The emitter may also be a toroidal (i.e., donut-shaped) permanent magnet.

In another embodiment of the invention, the magnetic therapy device may include a magnetic therapy system for beneficial use of a person and/or other user. There may be a reclining surface with one or more supporting legs. There may be one or more magnetic flux emitters, Each emitter may be placed about a different leg, and/or each emitter may be oriented in substantially the same direction. The reclining surface may be a bed.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

### BRIEF DESCRIPTION OF THE DRAWINGS

In order for the advantages of the invention to be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 illustrates a schematic side view of one embodiment of the invention.

FIG. 2 illustrates an isometric sectional close-up view of one leg of a bed and an emitter according to one embodiment of the invention.

### DETAILED DESCRIPTION OF THE INVENTION

For the purposes of promoting an understanding of the principles of the invention, reference will now be made to the exemplary embodiments illustrated in the drawings, and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would occur to one skilled in the relevant art and having possession of this disclosure, are to be considered within the scope of the invention.

Reference throughout this specification to “one embodiment,” “an embodiment,” or similar language means that a particular feature, structure, or characteristic described in connection with the embodiment is included in at least one embodiment of the present invention. Thus, appearances of the phrases “one embodiment,” “an embodiment,” and similar language throughout this specification may, but do not necessarily, all refer to the same embodiment, different embodiments, or component parts of the same or different illustrated invention. Additionally, reference to the wording “an embodiment,” or the like, for two or more features, elements, etc. does not mean that the features are related, dissimilar, the same, etc. The use of the term “an embodiment,” or similar wording, is merely a convenient phrase to indicate optional features, which may or may not be part of the invention as claimed.

Each statement of an embodiment is to be considered independent of any other statement of an embodiment despite any use of similar or identical language characterizing each embodiment. Therefore, where one embodiment is identified as “another embodiment,” the identified embodiment is independent of any other embodiments characterized by the language “another embodiment.” The independent embodiments are considered to be able to be combined in whole or in part one with another as the claims and/or art may direct, either directly or indirectly, implicitly or explicitly.

Finally, the fact that the wording “an embodiment,” or the like, does not appear at the beginning of every sentence in the specification, such as is the practice of some practitioners, is merely a convenience for the reader's clarity. However, it is the intention of this application to incorporate by reference the phrasing “an embodiment,” and the like, at the beginning of every sentence herein where logically possible and appropriate.

For the purpose of describing this embodiment, the flux lines emitting from an emitter and/or magnet are described as having a positive and negative orientation. Specifically, flux emitting from the north pole is corresponding to a positive flux, and the opposite from the south pole, i.e. it being considered negative flux.

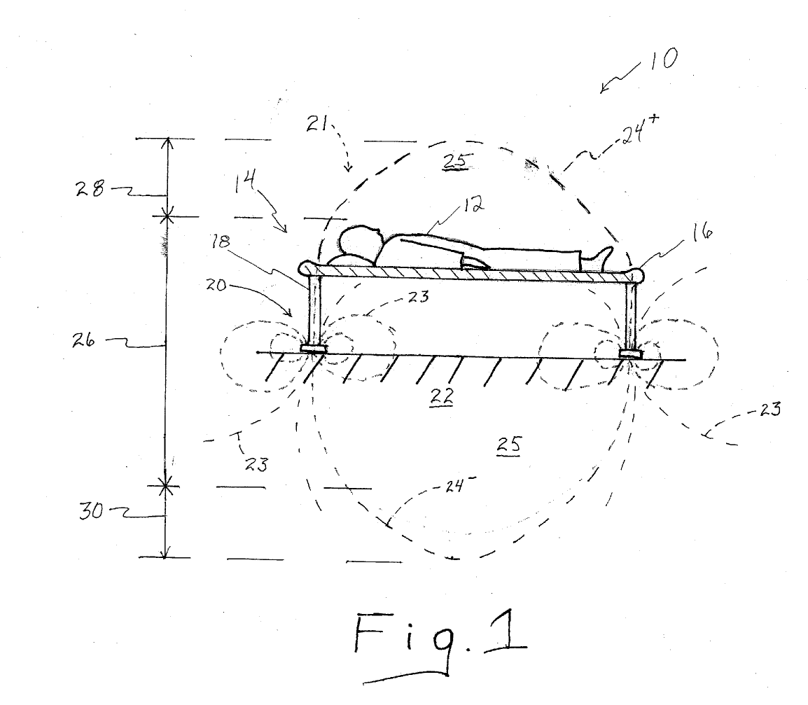

FIG. 1 illustrates a schematic side view of one embodiment of the invention. Specifically, there is illustrated one embodiment with a reclining user 12 in a body-neutralizer system 10 that may include a bed 14, which is positioned on a floor 22. The bed 14 typically has four legs 18 that are typically used for stabilization and a reclining surface 16 typically used for cushioning the user 12. Placed at each of the legs 18 is a magnetic flux emitter 20, which may be in the form of a magnet. The emitter 20 may emit a magnetic flux field 21 that includes flux lines 23. Additionally, it is theorized that, as illustrated, there is a net effect or amalgamation of the flux fields from all of the emitters, representatively shown as an effective shell 24. As illustrated, it is theorized that the effective shell 24 may have an positive upper section (24+) and/or a negative lower section (24−), and this shell may create a magnetic barrier/shield and an envelope area 25 (also, created by the shell 24), which envelopes the user 12 during use of the bed 14.

Due to the orientation of the flux field 21, and as a result of having all of the emitters 20 oriented in the same direction (e.g. north pointing upward), there are three regions created: Namely, there is a positive region or zone 28, a neutral zone 26, and a negative zone 30. Note, the positive and negative zones may be reversed by reversing the polarity orientation of the emitters 20, while still maintaining a neutral zone 26 about the user 12. For example, and by way of illustration only, the positive zone may be zone 28, which is created by having a predominant positive direction to the flux lines based on the north poles of the emitters being orientated upward, and the south poles all pointing downward, thus, creating the negative zone 30.

It is theorized that by placing the person(s) and/or animal(s) within the neutral zone within the effective shield/envelope, health benefits are derived. However, the magnetic therapy device may play other roles, and therefore the shield/envelope and/or neutral zone theories are not intended to be limiting to the invention.

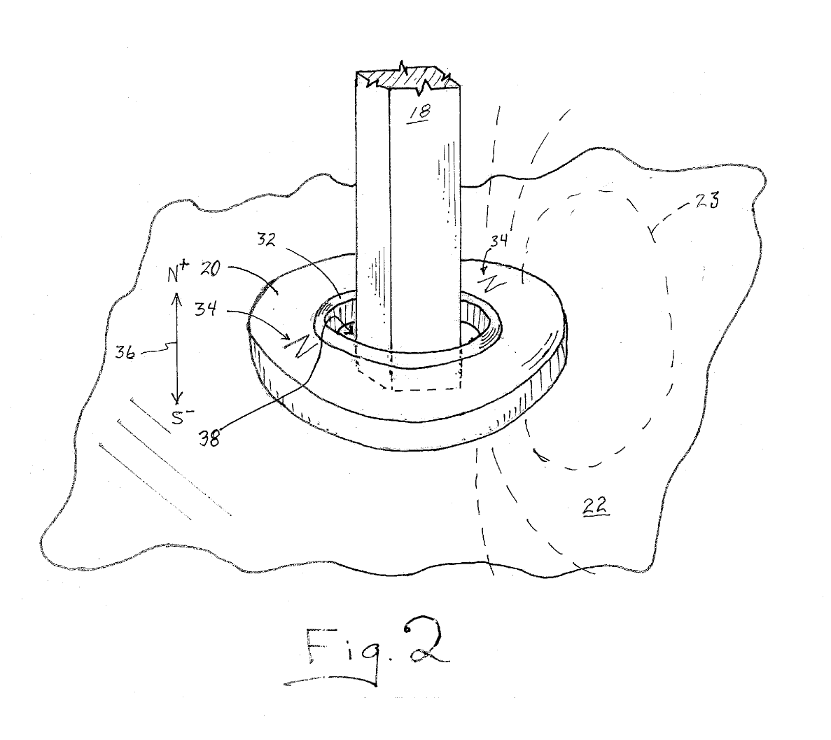

FIG. 2 illustrates an isometric sectional close-up view of one embodiment of the invention. Specifically, there is one leg of a bed 18 and an emitter 20 according to one embodiment of the invention. In addition to the elements labeled from the previous figure, in one embodiment, the emitter 20 may include a protective layer 32 to prevent scratching and/or marring and/or other damage to the leg and/or other supporting structure 18. Additionally, there may be an indicator 34, which is illustrated as the “N” for north pole of the emitter 20. Polarity orientation line 36 (N+—S−) indicates a default preference for all of the emitters to be oriented or aligned in the same direction to create and optimize the proper zones 26, 28 and 30, although there may be other desirable combinations of orientations. There may also be an aperture 38 through which the leg 18 may pass for several reasons. The aperture 38 may be for the purpose of securing the emitter 20 to the bed 14 and/or other frame 14; the aperture 38 may also serve to position each emitter 20 in a place relative to the overall system 10 so as to be evenly dispersed about the system 10, so as to provide the optimal magnetic field and thus the optimal health shield/envelope. Aperture 38 may also be a recessed area like a cup that doesn't pass entirely through the mass of emitter 20, such that it serves as a coaster to the leg 18 as well. There may be other uses of the emitter(s) and its aperture(s) as well.

It is understood that the above-described embodiments are only illustrative of the application of the principles of the present invention. The present invention may be embodied in other specific forms without departing from its spirit or essential characteristics. The described embodiment is to be considered in all respects only as illustrative and not restrictive. The scope of the invention is, therefore, indicated by the appended claims rather than by the foregoing description. All changes which come within the meaning and range of equivalency of the claims are to be embraced within their scope.

Although specific depiction of certain elements is provided in the accompanied drawings, it is to be understood that the drawings are merely illustrated. For example, emitter 20 is illustrated as a circular donut (i.e., toroidal) permanent magnet with a center hole (aperture) large enough to contain a leg 18 of a bed 14. Nevertheless, any shape may be used to effectuate the three zones 26, 28 and 30. For example, a triangle, half circle, cube, cat, dog, etc. would all be sufficient designs.

Additionally, there is illustrated a protection layer 32 that is positioned only on the inside layer of the emitter 20. It may be useful to have the protection layer 32 located completely around the entire surface of the emitter 20, thus encapsulating the emitter 20. Similarly, there is illustrated an indicator 34, which indicates what surface is north or south polarity. Specifically, there is illustrated a “N” symbol, nevertheless, any type of indication may be utilized. For example, the entire top surface may be colored red to indicate which face is the north face. Thus, any indicator method may be used to assist the user in placing all of the emitters 20 in the same orientation.

It is noted that the bed 14 is illustrated to have a certain height off of the floor 22, and thus being somewhat removed from the placement of the emitters 20. This orientation places the user 12 in an upper region of the neutral zone 26. It is actually envisioned to situate the user 12 anywhere within the neutral zone 26, and not just in the upper region as shown. Thus, the bed legs 18 may be shorter, or the emitters may be elevated off of the floor to position them closer to the user. Obviously, each of the emitters would need to be positioned relatively in the same horizontal plane for maximum effect to the flux field.

Although the emitters 20 are illustrated as being of uniform material, most any form of emitter 20 may be utilized to create the necessary flux field 21. For example, there may be electromagnets placed around the user 12, and the user may turn them on during resting states, and deactivate when not being used. Additionally, the emitters may be most any thickness, hardness, intensity, material composition, etc. The illustrated embodiment illustrates the bed 14 having legs 18. The legs 18, and maybe including a frame of the bed, may be constructed with magnetically conductive or non-conductive material, and may be of varying number, shape, etc. There may be one or more legs 18 (i.e., a plurality of bed legs); there may be one or more magnetic flux emitters 20 (i.e., a plurality of magnetic flux emitters). The magnetic flux emitters 20 may be oriented in the same direction; and/or their orientations may be mixed in any combination of possibilities to provide various field effects 21.

The reclining user 12 may also be more than one person and/or may be an animal and/or more than one animal, separately or simultaneously. The position of the person(s) and/or animal(s) 12 may be reclining, lying down, sitting, kneeling, and/or any other position which may be desirable.

It is understood that the above-described embodiments are only illustrative of the application of the principles of the present invention. The present invention may be embodied in other specific forms without departing from its spirit or essential characteristics. The described embodiment is to be considered in all respects only as illustrative and not restrictive. The scope of the invention is, therefore, indicated by the appended claims rather than by the foregoing description. All changes which come within the meaning and range of equivalency of the claims are to be embraced within their scope.

Thus, while the present invention has been fully described above with particularity and detail in connection with what is presently deemed to be the most practical and preferred embodiment of the invention, it will be apparent to those of ordinary skill in the art that numerous modifications, including, but not limited to, variations in size, materials, shape, form, function and manner of operation, assembly and use may be made, without departing from the principles and concepts of the invention as set forth in the claims.

What is claimed is:

1. A magnetic device for therapy of humans and animals, comprising:
a magnetic flux emitter including an aperture therethrough; and
a protective layer disposed in the aperture.

2. The magnetic device of claim 1, further comprising an indicator disposed on the magnetic flux emitter and configured to indicate orientation of flux.

3. The magnetic device of claim 2, wherein the magnetic flux emitter comprises a toroidal permanent magnet.

4. A magnetic therapy system for beneficial use of a person, comprising:
a reclining surface;
a plurality of supporting legs coupled to and supporting the reclining surface; and
a plurality of magnetic flux emitters, each disposed about one of the plurality of supporting legs and each oriented in substantially the same direction.

5. The magnetic therapy system of claim 4, wherein the reclining surface is a bed.

6. The magnetic therapy system of claim 5, wherein the plurality of magnetic flux emitters each include a protective layer configured to prevent damage to the plurality of supporting legs by contact with the plurality of magnetic flux emitters.

7. A magnetic device for therapy of humans and animals, consisting essentially of:
a magnetic flux emitter including an aperture therethrough; and
a protective layer disposed in the aperture.

8. The magnetic device of claim 7, further comprising an indicator disposed on the magnetic flux emitter and configured to indicate orientation of flux.

9. The magnetic device of claim 8, wherein the magnetic flux emitter comprises a toroidal permanent magnet.
